(19) United States
(12) Patent Application Publication
(10) Pub. No.: US 2004/0152720 A1
(43) Pub. Date: Aug. 5, 2004

(54) Powdered medicaments containing a tiotropium salt and salmeterol xinafoate

(75) Inventors: Mareke Hartig, Ingelheim (DE); Michael Trunk, Ingelheim (DE); Rainer Soyka, Biberach (DE); Peter Sieger, Mittelbiberach (DE); Hagen Graebner, Ingelheim (DE); Michael Walz, Bingen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/736,264

(22) Filed: Dec. 15, 2003

Related U.S. Application Data
(60) Provisional application No. 60/446,670, filed on Feb. 11, 2003.

(30) Foreign Application Priority Data
Dec. 20, 2002 (DE) DE 102 59 912

(51) Int. Cl. A61K 31/4745; A61K 31/137; A61K 31/205

(52) U.S. Cl. 514/291; 514/554

(57) ABSTRACT

The invention relates to powdered preparations for inhalation containing a tiotropium salt and salmeterol xinafoate, processes for preparing them and their use in the preparation of a pharmaceutical composition for treating respiratory diseases, particularly for treating COPD (chronic obstructive pulmonary disease) and asthma.

### RELATED APPLICATIONS

Benefit of U.S. Provisional Application Serial No. 60/446,670, filed on Febr. 11, 2003 is hereby claimed.

### FIELD OF THE INVENTION

The invention relates to powdered preparations for inhalation containing a tiotropium salt and salmeterol xinafoate, processes for preparing them and their use in the preparation of a pharmaceutical composition for treating respiratory diseases, particularly for treating COPD (chronic obstructive pulmonary disease) and asthma.

### BACKGROUND TO THE INVENTION

Tiotropium bromide is known from European Patent Application EP 418 716 A1 and has the following chemical structure:

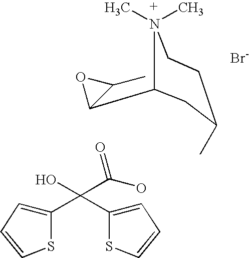

Tiotropium bromide, like the other salts of tiotropium, is a highly effective anticholinergic with a long-lasting activity which can be used to treat respiratory complaints, particularly COPD (chronic obstructive pulmonary disease) and asthma. The term tiotropium refers to the free ammonium cation.

The betamimetic salmeterol is also known from the prior art. It is used for example in the treatment of asthma.

WO 00/69468 discloses pharmaceutical combinations of long-acting betamimetics with long-acting anticholinergics which are characterised by the synergistic effect of the two pharmaceutical ingredients. One specific pharmaceutical combination disclosed in WO 00/69468 is the combination of tiotropium bromide and salmeterol xinafoate.

The active substances salmeterol and tiotropium are administered by inhalation. Suitable inhalable powders may be used.

The correct manufacture of the abovementioned compositions which may be used for the administration by inhalation of a pharmaceutical active substance is based on various parameters connected with the nature of the pharmaceutical active substance itself. Without being restricted thereto, examples of these parameters are the stability of effect of the starting material under different ambient conditions, the stability during the manufacture of the pharmaceutical formulation and the stability in the finished compositions of the pharmaceutical substance. The active substance used to prepare the pharmaceutical compositions mentioned above should be as pure as possible, and its stability during long-term storage should be guaranteed under a variety of ambient conditions. This is absolutely essential to prevent the use of pharmaceutical compositions which contain breakdown products of the active substance as well as the active substance itself. In such a case the content of active substance in a capsule might be lower than specified.

Uniform distribution of the drug in the formulation is also a critical factor, particularly when a low dose of the drug is needed. This is particularly important when a mixture of active substances is to be used. Another aspect which is important in the case of active substances to be administered by inhalation of a powder stems from the fact that only particles of a certain particle size are able to reach the lungs during inhalation. The particle size of these lung-bound particles (inhalable proportion) is in the sub-micron range. In order to obtain active substances of the requisite particle size, a grinding process (so-called micronising) is also required.

As any breakdown of the pharmaceutically active substance as a side effect of the grinding (or micronising) has to be avoided as far as possible, in spite of the hard conditions required in the course of the process, high stability of the active substance in the grinding process is absolutely essential. Only if the active substance is sufficiently stable during the grinding process is it possible to produce a homogeneous pharmaceutical formulation which will always contain the specified amount of active substance in a reproducible manner. Another problem that may arise in the grinding process for producing the desired pharmaceutical formulation is the input of energy caused by this process and the stress on the surface of the crystals. In some cases this may lead to polymorphic changes, a change in the amorphous structure or an alteration in the crystal lattice. As it is essential to maintain the same crystalline morphology for the active substance at all times to ensure the pharmaceutical quality of a pharmaceutical formulation, the stability and properties of the crystalline active substance are also subject to very stringent requirements against this background.

In addition to the requirements outlined above it should be generally remembered that any change in the solid state of a drug which is capable of improving its physical and chemical stability confers a substantial advantage over less stable forms of the same drug.

The aim of the present invention is to provide a pharmaceutical formulation containing a tiotropium salt and salmeterol xinafoate wherein both active substances satisfy the requirements mentioned above. In particular, the invention also sets out to provide a pharmaceutical formulation containing a tiotropium salt and salmeterol xinafoate which is characterised by the maximum possible stability of the two active substances in the formulation.

The active substances tiotropium and salmeterol are particularly effective. With active substances which have a particularly high efficacy, only small amounts of the active substance are needed per single dose to achieve the desired therapeutic effect. In such cases, the active substance has to be diluted with suitable excipients in order to prepare the inhalable powder. Because of the large amount of excipient, the properties of the inhalable powder are critically influenced by the choice of excipient. When choosing the excipient its particle size is particularly important. As a rule, the finer the excipient, the poorer its flow properties. However, good flow properties are a prerequisite for highly accurate metering when packing and dividing up the individual doses of preparation, e.g. when producing capsules (inhalettes) for powder inhalation or when the patient is metering the individual dose before using a multi-dose inhaler. Moreover, the particle size of the excipient is very important for the emptying characteristics of capsules when used in an inhaler. It has also been found that the particle size of the excipient has a considerable influence on the proportion of active substance in the inhalable powder which is delivered for inhalation. The term inhalable proportion of active substance refers to the particles of the inhalable powder which are conveyed deep into the branches of the lungs when inhaled with a breath. The particle size required for this is between 1 and 10 μm, preferably less than 6 μm.

The aim of the invention is to prepare an inhalable powder containing a tiotropium salt and salmeterol xinafoate which, while being accurately metered (in terms of the amount of active substance and powder mixture packed into each capsule by the manufacturer as well as the quantity of active substance released and delivered to the lungs from each capsule by the inhalation process) with only slight variations between batches, enables the active substance to be administered in a large inhalable proportion. A further aim of the present invention is to prepare an inhalable powder containing a tiotropium salt and salmeterol xinafoate which ensures good emptying characteristics of the capsules, whether it is administered to the patient using an inhaler, for example, as described in WO 94/28958, or in vitro using an impactor or impinger.

The fact that tiotropium salts, but also particularly salmeterol xinafoate, have a high therapeutic efficacy even at very low doses imposes further conditions on an inhalable powder containing two of the abovementioned active substances which is to be used with highly accurate metering. Because only a low concentration of the active substances is needed in the inhalable powder to achieve the therapeutic effect, a high degree of homogeneity of the powder mixture and only slight fluctuations in the dispersion characteristics from one batch of capsules to the next are essential. The homogeneity of the powder mixture and minor fluctuations in the dispersion properties are crucial in ensuring that the inhalable proportion of the active substances is released reproducibly in constant amounts and with the lowest possible variability.

Accordingly, a further aim of the present invention is to prepare an inhalable powder containing a tiotropium salt and salmeterol xinafoate which is characterised by a high degree of homogeneity and uniformity of dispersion. The present invention also sets out to provide an inhalable powder which allows the inhalable proportion of active substance to be administered with the lowest possible variability.

The characteristics of emptying from the powder reservoir (the container from which the inhalable powder containing the active substances is released for inhalation) play an important part, not exclusively, but especially in the administration of inhalable powders using capsules containing powder. If only a small amount of the powder formulation is released from the powder reservoir as a result of minimal or poor emptying characteristics, significant amounts of the inhalable powder containing the active substances are left in the powder reservoir (e.g. the capsule) and are unavailable to the patient for therapeutic use. The result of this is that the dosage of active substance in the powder mixture has to be increased so that the quantity of active substance delivered is sufficient to produce the desired therapeutic effect.

Against this background the present invention further sets out to provide an inhalable powder containing a tiotropium salt and salmeterol xinafoate which is also characterised by very good emptying characteristics.

### DETAILED DESCRIPTION OF THE INVENTION

It was found that, surprisingly, the objectives outlined above can be achieved by means of the powdered preparations for inhalation (inhalable powders) according to the invention described hereinafter containing a tiotropium salt 1 and salmeterol xinafoate 2.

Within the scope of the present invention the term tiotropium salts 1 denotes salts which are formed from the pharmacologically active cation tiotropium 1′. Within the scope of the present patent application an explicit reference to the cation tiotropium is indicated by the use of the designation 1′.

The inhalable powders according to the invention contain tiotropium 1′ and salmeterol xinafoate 2, which is characterised by a melting point of about 124° C., mixed with a physiologically acceptable excipient.

The melting point mentioned above was obtained by DSC (Differential Scanning Calorimetry) using a Mettler DSC 820 and evaluated using the Mettler STAR software package. The data was recorded at a heating rate of 10 K/min.

Preferably, the salmeterol xinafoate 2 used in the inhalable powders according to the invention has the following characteristic values, inter alia, in the X-ray powder diagram: d=21.5 Å; 8.41 Å; 5.14 Å; 4.35 Å; 4.01 Å and 3.63 Å.

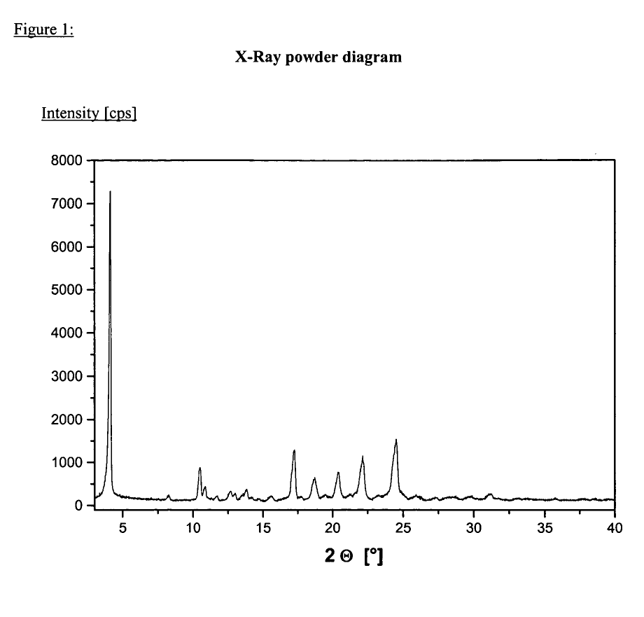

Detailed information on the method of obtaining the characteristic X-ray powder diagram data can be found in the experimental section of the present invention. The X-ray powder diagram of the salmeterol xinafoate which is preferably used according to the invention is shown in FIG. 1.

Particularly preferably, the salmeterol xinafoate 2 used in the inhalable powders according to the invention has a compacted bulk volume of≧0.134 g/cm³, preferably≧0.14 g/cm³, particularly preferably≧0.145 g/cm³.

The compacted bulk volume is determined by the test method of the European Pharmacopoeia 4 (2002): “apparent density after settling”/“density of settled product”, identical to the “tapped density”, measured in grams per millilitre) or as the “Carr packed bulk density” according to the ASTM standard (D6393-99, Standard Test Method for Bulk Solids Characterization by Carr Indices), measured in grams per cm³. The compacted bulk volume is a measurement of the volume which is taken up by solid, comminuted materials after they have been compacted under defined conditions.

The particular suitability of salmeterol xinafoate, which is characterised by the above parameters, applies both to the starting material of a micronising process and to the use of a micronised preparation of this substance with the above physical properties in the course of the manufacture of an inhalable powder. In particular, both the product obtained after micronisation and the salmeterol xinafoate used in the micronisation are characterised by the above-mentioned parameters.

In the inhalable powders according to the invention the salmeterol xinafoate 2 described above is preferably present in an amount of from 0.002 to 15%. Inhalable powders which contain 0.01 to 10% of 2 are preferred according to the invention. Particularly preferred inhalable powders contain 2 in an amount of from 0.05 to 5%, preferably 0.1 to 3%, particularly preferably 0.125 to 2%, still more preferably 0.25 to 2%.

The inhalable powders according to the invention also preferably contain 0.001 to 5% tiotropium 1′. Preferred according to the invention are inhalable powders which contain 0.01 to 3% tiotropium 1′. Particularly preferred inhalable powders contain tiotropium 1′ in an amount of 0.02 to 2.5%, preferably 0.03 to 2.5%, particularly preferably 0.04 to 2%.

By tiotropium 1′ is meant the free ammonium cation. Where the term 1 is used within the scope of the present invention, this should be interpreted as being a reference to tiotropium combined with a corresponding counter-ion. The counter-ion (anion) may preferably be chloride, bromide, iodide, methanesulphonate or para-toluenesulphonate. Of these anions, the bromide is preferred.

Accordingly, the present invention preferably relates to inhalable powders which contain between 0.0012 and 6%, preferably 0.012 to 3.6% tiotropium bromide 1. Of particular interest according to the invention are inhalable powders which contain about 0.024 to 3%, preferably about 0.036 to 3%, particularly preferably about 0.048 to 2.4% tiotropium bromide 1.

The tiotropium bromide which is preferably contained in the inhalable powders according to the invention may include solvent molecules during crystallisation. Preferably, the hydrates of tiotropium bromide are used to prepare the tiotropium-containing inhalable powders according to the invention. Most preferably, the crystalline tiotropium bromide monohydrate known from WO 02/30928 is used. This crystalline tiotropium bromide monohydrate is characterised by an endothermic maximum at 230±5° C. at a heating rate of 10K/min, when thermally analysed by DSC. It is also characterised in that in the IR spectrum it has bands inter alia at wavelengths 3570, 3410, 3105, 1730, 1260, 1035 and 720 cm⁻¹. Finally, this crystalline tiotropium bromide monohydrate has a simple monoclinic cell with the following dimensions: a=18.0774 Å, b=11.9711 Å, c=9.9321 Å, β=102.691°, V=2096.96 Å³ as determined by monocrystalline X-ray structural analysis.

Accordingly the present invention relates to powders for inhalation which contain between 0.00125 to 6.25%, preferably 0.0125 to 3.75% of crystalline tiotropium bromide monohydrate. Of particular interest according to the invention are inhalable powders which contain about 0.025 to 3.125%, preferably about 0.0375 to 3.125%, particularly preferably about 0.05 to 2.5% of tiotropium bromide monohydrate.

The percentages given within the scope of the present invention are always percent by weight, unless specifically stated to the contrary.

The pharmaceutical compositions according to the invention containing the combinations of 1 and 2 are usually administered so that each single dose contains a combination of tiotropium 1′ and salmeterol xinafoate 2 in doses from 5 to 5000 μg, preferably from 10 to 2000 μg, particularly preferably from 15 to 1000 μg, still more preferably from 20 to 500 μg, preferably according to the invention from 25 to 250 μg, preferably 30 to 125 [μg, particularly preferably from 40 to 70 μg.

For example, and without restricting the scope of the invention thereto, the combinations of 1 and 2 according to the invention may contain an amount of tiotropium 1′ and salmeterol xinafoate 2 such that for example 4.5 μg of 1 and 25 μg of 2, 4.5 μg of 1′ and 30 μg of 2, 4.5 μg of 1′ and 35 μg of 2, 4.5 μg of 1′ and 40 μg of 2, 4.5 μg of 1′ and 43.5 μg of 2, 4.5 μg of 1 and 50 μg of 2, 4.5 μg of 1′ and 60 μg of 2, 4.5 μg of 1′ and 70 μg of 2, 4.5 μg of 1′ and 80 μg of 2, 4.5 μg of 1′ and 90 μg of 2, 4.5 μg of 1′ and 100 μg of 2, 4.5 μg of 1′ and 110 μg of 2, 10 μg of 1′ and 25 μg of 2, 10 μg of 1′ and 30 μg of 2, 10 μg of 1′ and 35 μg of 2, 10 μg of 1 and 40 μg of 2, 10 μg of 1′ and 50 μg of 2, 10 μg of 1′ and 60 μg of 2, 10 μg of 1′ and 70 μg of 2, 10 μg of 1′ and 80 μg of 10 μg of 1′ and 90 μg of 2, 10 μg of 1′ and 100 μg of 2, 10 μg of 1′ and 110 μg of 2, 18 μg of 1′ and 25 μg of 2, 18 μg of 1′ and 30 μg of 2, 18 μg of 1′ and 35 μg of 2, 18 μg of 1′ and 40 μg of 2, 18 μg of 1′ and 50 μg of 2, 18 μg of 1′ and 60 μg of 2, 18 μg of 1′ and 70 μg of 2, 18 μg of 1′ and 80 μg of 2, 18 μg of 1′ and 90 μg of 2, 18 μg of 1′ and 100 μg of 2, 18 μg of 1′ and 110 μg of 2, 36 μg of 1′ and 25 μg of 2, 36 μg of 1′ and 30 μg of 2, 36 μg of 1′ and 35 μg of 2, 36 μg of 1′ and 40 μg of 2, 36 μg of 1′ and 50 μg of 2, 36 μg of 1′ and 60 μg of 2, 36 μg of 1′ and 70 μg of 2, 36 μg of 1′ and 80 μg of 2, 36 μg of 1′ and 90 μg of 2, 36 μg of 1′ and 100 μg of 2, 36 μg of 1′ and 110 μg of 2 are administered per single dose.

If the active substance combination wherein the bromide is used as the salt 1 is used as a preferred combination of 1 and 2 according to the invention, the quantities of active substances 1′ and 2 administered per single dose mentioned above by way of example correspond approximately to the following amounts of 1 and 2 administered per single dose: 5.4 μg of 1 and 25 μg of 2, 5.4 μg of 1 and 30 μg of 2, 5.4 μg of 1 and 35 μg of 2, 5.4 μg of 1 and 40 μg of 2, 5.4 μg of 1 and 50 μg of 2, 5.4 μg of 1′ and 60 μg of 2, 5.4 μg of 1 and 70 μg of 2, 5.4 μg of 1 and 80 μg of 2, 5.4 μg of 1 and 90 μg of 2, 5.4 μg of 1 and 100 μg of 2, 5.4 μg of 1 and 110 μg of 2, 12 μg of 1 and 25 μg of 2, 12 μg of 1 and 30 μg of 2, 12 μg of 1 and 35 μg of 2, 12 μg of 1 and 40 μg of 2, 12 μg of 1 and 50 μg of 2, 12 μg of 1 and 60 μg of 2, 12 μg of 1 and 70 μg of 2, 12 μg of 1 and 80 μg of 2, 12 μg of 1 and 90 μg of 2, 12 μg of 1 and 100 μg of 2, 12 μg of 1 and 100 μg of 2, 21.7 μg of 1 and 25 μg of 2, 21.7 μg of 1 and 30 μg of 2, 21.7 μg of 1 and 35 μg of 2, 21.7 μg of 1 and 40 μg of 2, 21.71 μg of 1 and 50 μg of 2, 21.7 μg of 1 and 60 μg of 2, 21.7 μg of 1 and 70 μg of 2, 21.7 μg of 1 and 80 μg of 2, 21.7 μg of 1 and 90 μg of 2, 21.7 μg of 1 and 100 μg of 2, 21.7 μg of 1 and 110 μg of 2, 43.3 μg of 1 and 25 μg of 2, 43.3 μg of 1 and 30 μg of 2, 43.3 μg of 1 and 35 μg of 2, 43.3 μg of 1 and 40 μg of 2, 43.3 μg of 1 and 50 μg of 2, 43.3 μg of 1 and 60 μg of 2, 43.3 μg of 1 and 70 μg of 2, 43.3 μg of 1 and 80 μg of 2, 43.3 μg of 1 and 90 μg of 2, 43.3 μg of 1 and 100 μg of 2, 43.3 μg of 1 and 100 μg of 2.

If the active substance combination wherein the crystalline tiotropium bromide monohydrate is used as the salt 1 is used as a preferred combination of 1 and 2 according to the invention, the quantities of active substances 1 and 2 mentioned above by way of example administered per single dose correspond approximately to the following amounts of tiotropium bromide monohydrate 1 and 2 administered per single dose: 5.6 μg of 1 and 25 μg of 2, 5.6 μg of 1 and 30 μg of 2, 5.6 μg of 1 and 35 μg of 2, 5.6 μg of 1 and 40 μg of 2, 5.6 μg of 1 and 50 μg of 2, 5.6 μg of 1 and 60 μg of 2, 5.6 μg of a and 70 μg of 2, 5.6 μg of 1 and 80 μg of 2, 5.6 μg of 1 and 90 μg of 2, 5.6 μg of a and 100 μg of 2, 5.6 μg of 1 and 110 μg of 2, 12.5 μg of 1 and 25 μg of 2, 12.5 μg of 1 and 30 μg of 2, 12.5 μg of 1 and 35 μg of 2, 12.5 μg of 1 and 40 μg of 2, 12.5 μg of 1 and 50 μg of 2, 12.51 μg of 1 and 60 μg of 2, 12.5 μg of 1 and 70 μg of 2, 12.5 μg of 1 and 80 μg of 2, 12.5 μg of 1 and 90 μg of 2, 12.5 μg of 1 and 100 μg of 2, 12.5 μg of 1 and 110 μg of 2, 22.5 μg of 1 and 25 μg of 2, 22.5 μg of 1 and 30 μg of 2, 22.5 μg of 1 and 35 μg of 2, 22.5 μg of 1 and 40 μg of 2, 22.5 μg of 1 and 50 μg of 2, 22.5 μg of 1 and 60 μg of 2, 22.5 μg of 1 and 70 μg of 2, 22.5 μg of 1 and 80 μg of 2, 22.5 μg of 1 and 90 μg of 2, 22.5 μg of 1 and 100 μg of 2, 22.5 μg of 1 and 110 μg of 2, 45 μg of 1 and 25 μg of 2, 45 μg of 1 and 30 μg of 2, 45 μg of 1 and 35 μg of 2, 45 μg of 1 and 40 μg of 2, 45 μg of 1 and 50 μg of 2, 45 μg of 1 and 60 μg of 2, 45 μg of 1 and 70 μg of 2, 45 μg of 1 and 80 μg of 2, 45 μg of 1 and 90 μg of 2, 45 μg of 1 and 100 μg of 2, 45 μg of 1 and 110 μg of 2.

Examples of physiologically acceptable excipients which may be used to prepare the inhalable powders used in the drug preparations according to the invention include, for example, monosaccharides (e.g. glucose or arabinose), disaccharides (e.g. lactose, saccharose, maltose, trehalose), oligo- and polysaccharides (e.g. dextrane), polyalcohols (e.g. sorbitol, mannitol, xylitol), or salts (e.g. sodium chloride, calcium carbonate). Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Particularly preferably, excipients are used which have an average particle size of 10-50 μm. By the average particle size is meant here the 50% value of the volume distribution measured using a laser diffractometer by the dry dispersion method. In particularly preferred inhalable powders the excipient is characterised by an average particle size of 12 to 35 μm, particularly preferably 13 to 30 μm.

It is also particularly preferable to use excipients which have a 10% fine content of from 0.5 to 6 μm. The 10% fine content in this instance refers to the 10% value of the volume distribution measured using a laser diffractometer. In other words, for the purposes of the present invention, the 10% fine content denotes the particle size below which 10% of the quantity of particles is found (based on the volume distribution). Also particularly preferred are those inhalable powders wherein the 10% fine content is about 1 to 4 μm, preferably about 1.5 to 3 μm.

Also preferred according to the invention are those inhalable powders wherein the excipient has a specific surface area of between 0.2 and 1.5 m²/g, preferably between 0.3 and 1.0 m²/g.

Preferably, excipients of high crystallinity are used for the powder formulations according to the invention. This crystallinity can be assessed by means of the enthalpy released as the excipient is dissolved (solution enthalpy). In the case of the excipient lactose monohydrate, which is most preferably used according to the invention, it is preferable to use lactose which is characterised by a solution enthalpy of ≧45 J/g, preferably ≧50 J/g, particularly preferably ≧52 J/g.

The inhalable powders according to the invention are characterised, in accordance with the problem on which the invention is based, by a high degree of homogeneity in the sense of the accuracy of single doses. This is in the region of <8% , preferably <6%, most preferably <4%.

In some cases it may be helpful to use, as an alternative to the excipients mentioned above, excipient mixtures consisting of a mixture of coarser excipient with an average particle size of 17 to 50 μm, preferably 20 to 40 μm, more preferably 25 to 35 μm and finer excipient with an average particle size of 1 to 8 μm, preferably 2 to 7 μm, particularly preferably 3 to 6 μm . Here again, by the average particle size is meant the 50% value from the volume distribution measured by laser diffraction according to the dry dispersion method.

If the above-mentioned excipient mixtures are used, the 10% fine content of the coarser excipient component is about 2 to 5 μm, preferably about 3 to 4 μm, and that of the finer excipient component is about 0.5 to 1.5 μm.

Inhalable powders are preferred wherein the proportion of finer excipient in the total formulation is 2 to 10%, preferably 3 to 7%, particularly preferably 4 to 6%. Where reference is made to the term “excipient mixture” within the scope of the present invention, this is always a mixture obtained by mixing components which have previously been clearly defined. Accordingly, where reference is made to an excipient mixture of coarser and finer excipient ingredients, only those mixtures are meant which have been obtained by mixing a coarser excipient component with a finer excipient component. The coarser and finer excipient fractions may consist of chemically identical or chemically different substances selected from the substances mentioned earlier as examples of excipients, while inhalable powders in which the coarser excipient component and the finer excipient component consist of the same chemical compound are preferred. If for example lactose monohydrate is used as excipient, when an excipient fraction with a smaller average particle size as described above is specifically added, this is preferably also lactose monohydrate.

In order to prepare the pharmaceutical compositions according to the invention it is first necessary to prepare salmeterol xinafoate 2 in a form which satisfies the abovementioned specifications for 2.

According to the invention, the following procedure is preferably adopted.

The free base of salmeterol known from the prior art is taken up together with 1-hydroxy-2-naphthoic acid in a solvent mixture consisting of an alcohol and an ether. For each mol of salmeterol used, at least 1 mol of 1-hydroxy-2-naphthoic acid, preferably 1 to 1.1 mol of 1-hydroxy-2-naphthoic acid, most preferably 1 mol of 1-hydroxy-2-naphthoic acid is used. The alcohol which may be used according to the invention may be a lower-chained alcohol, preferably ethanol, n-propanol or isopropanol, particularly preferably ethanol. The ethers which may be used according to the invention are most preferably diethylether, methylethylether, tetrahydrofuran, dioxane or tert-butylmethylether, of which tert-butylmethylether is particularly preferred according to the invention. The ratio of alcohol to ether (ratio by volume) is preferably, according to the invention, in a range from about 1:2 to 2:1, particularly preferably in a range from about 1:1.5 to 1.5:1. Most preferably the ratio of alcohol to ether is 1:1.

The total amount of solvent used will naturally depend on the quantity of mixture. Preferably, about 5 to 20 litres, more preferably about 7 to 15 litres of solvent are used per mol of salmeterol base. Most preferably, about 9 to 12 litres of solvent are used per mol of salmeterol base, while in said solvents the two components alcohol and ether may be present in the ratios by volume specified above.

The suspension obtained is heated to a temperature of ≧40° C., preferably to a temperature of ≧50° C., most preferably to a temperature of about 55-56° C. after all the above-mentioned components have been added, and at the same time the suspension is stirred. Heating is continued until a clear solution is obtained. The solution is then filtered, and the filter is optionally rinsed out with a small amount (roughly 1 to 1.5 litres per mol of salmeterol used) of the solvent mentioned above. Then the filtrate obtained is cooled to a temperature of about 30 to 40° C., preferably about 35-38° C., and stirred at this temperature until the salmeterol xinafoate. begins to crystallise. It may be helpful at this point to add salmeterol xinafoate seed crystals. Once crystallisation has started the suspension is further cooled, with stirring, preferably to a temperature of about −10° C. to about 10° C., particularly preferably to a temperature of about 0° C. to about 5° C. After about 20 to 60 minutes crystallisation is complete and the product obtained is separated off using a suitable filter and optionally washed with alcohol and/or ether. The salmeterol xinafoate thus obtained complies with the abovementioned specification which characterises the inhalable powders according to the invention.

Accordingly, in another aspect, the present invention relates to inhalable powders containing, in addition to tiotropium 1′, salmeterol xinafoate 2 which can be obtained by the method described above.

After the starting materials have been weighed in the inhalable powders are prepared from the excipient and the active substance using methods known in the art. Reference may be made to the disclosure of WO 02/30390, for example. The inhalable powders according to the invention may accordingly be obtained by the method described below, for example. In the preparation methods described hereinafter the components are used in the proportions by weight described in the above-mentioned compositions of the inhalable powders.

First, the excipient and the tiotropium salt 1 are placed in a suitable mixing container. The active substance 1 used has an average particle size of 0.5 to 10 μm, preferably 1 to 6 μm, most preferably 2 to 5 μm. The excipient and the active substance 1 are preferably added using a sieve or a granulating sieve with a mesh size of 0.1 to 2 mm, preferably 0.3 to 1 mm, most preferably 0.3 to 0.6 mm. Preferably, the excipient is put in first and then the active substance is added to the mixing container. During this mixing process the two components are preferably added in batches. It is particularly preferred to sieve in the two components in alternate layers. The mixing of the excipient with the active substance 1 may take place while the two components are still being added. Preferably, however, mixing is only done once the two components have been sieved in layer by layer.

If the excipient used is an excipient mixture consisting of coarser excipient with an average particle size of from 17 to 50 μm, more preferably 20 to 35 μm, and finer excipient with an average particle size of 1 to 8 μm, more preferably 2 to 7 μm, preferably 3 to 6 μm, first of all the excipient mixture is prepared by sieving in alternate layers of the two excipient components and then mixing them together.

After the powder mixture described above containing the active substance 1 has been obtained, the salmeterol xinafoate 2 is added analogously. 2 also has an average particle size of 0.5 to 10 μm, preferably of 1 to 6 μm, more preferably 2 to 5 μm. 2 and the powder mixture containing component 1 are preferably added using a sieve or a granulating sieve with a mesh size of 0.1 to 2 mm, preferably 0.3 to 1 mm, most preferably 0.3 to 0.6 mm. Preferably, the powder mixture containing component 1 is put in first and then 2 is added to the mixing container. During this mixing process the two components are preferably added in batches. It is particularly preferred to sieve in the two components in alternate layers. The mixing of the powder mixture containing component 1 with the active substance 2 may take place while the two components are still being added. Preferably, however, mixing is only done once the two components have been sieved in layer by layer.

In an alternative embodiment of the invention, the inhalable powder according to the invention may also be obtained by first preparing a powder mixture consisting of excipient and 2 analogously to the method described above, to which component 1 is added by the method described above.

In another alternative embodiment of the invention, the inhalable powder according to the invention may also be obtained by first taking an excipient portion, then adding the first portion of 1 or the first portion of 2, then sieving in another excipient portion and finally adding the first portion of the second active substance component 1 or 2. This sequence of adding the excipient, substance 1 and substance 2 is then repeated until all the ingredients have been added in the desired amounts. It is also preferable to add the 3 components by sieving them in in alternate layers. The mixing process may take place while the 3 components are being added. Preferably, however, mixing is not done until all 3 components have been sieved in.

If after being chemically prepared the active substances 1 and 2 used in the process described above are not already obtainable in a crystalline form with the particle sizes mentioned earlier, they can be ground up into the particle sizes which conform to the above-mentioned parameters (so-called micronising). Suitable micronising methods are known from the prior art.

If the active substance 1 used is the crystalline tiotropium bromide monohydrate disclosed by WO 02/30928 which is particularly preferred according to the invention the following procedure has proved particularly suitable for micronising this crystalline active substance modification 1. The process may be carried out using conventional mills. Preferably, the micronisation is carried out with the exclusion of moisture, more preferably using a corresponding inert gas such as nitrogen, for example. It has proved particularly preferable to use air jet mills in which the material is comminuted by the impact of the particles on one another and on the walls of the grinding container. According to the invention, nitrogen is preferably used as the grinding gas. The material for grinding is conveyed by the grinding gas under specific pressures (grinding pressure).

Within the scope of the present invention, the grinding pressure is usually set to a value between about 2 and 8 bar, preferably between about 3 and 7 bar, most preferably between about 3.5 and 6.5 bar. The material for grinding is fed into the air jet mill by means of the feed gas under specific pressures (feed pressure). Within the scope of the present invention a feed pressure of between about 2 and 8 bar, preferably between about 3 and 7 bar and most preferably between about 3.5 and 6 bar has proved satisfactory. The feed gas used is also preferably an inert gas, most preferably nitrogen again. The material to be ground (crystalline tiotropium bromide monohydrate) may be fed in at a rate of about 5-35 g/min, preferably at about 10-30 g/min.

For example, without restricting the subject of the invention thereto, the following apparatus has proved suitable as a possible embodiment of an air jet mill: a 2-inch Microniser with grinding ring, 0.8 mm bore, made by Messrs Sturtevant Inc., 348 Circuit Street, Hanover, Mass. 02239, USA. Using this apparatus, the grinding process is preferably carried out with the following grinding parameters: grinding pressure: about 4.5-6.5 bar; feed pressure: about 4.5-6.5 bar; supply of grinding material: about 17-21 g/min.

The ground material thus obtained is then further processed under the following specific conditions. The micronisate is exposed to water vapour at a relative humidity of at least 40% at a temperature of 15-40° C., preferably 20-35° C., most preferably 25-30° C. Preferably, the humidity is set to a value of 50-95% r. h., preferably 60-90% r.h., most preferably 70-80% r.h. By relative humidity (r.h.) is meant the quotient of the partial steam pressure and the steam pressure of the water at the temperature in question. Preferably, the micronisate obtained from the grinding process described above is subjected to the chamber conditions mentioned above for a period of at least 6 hours. Preferably, however, the micronisate is subjected to the chamber conditions mentioned above for about 12 to 48 hours, preferably about 18 to 36 hours, more preferably about 20 to 28 hours.

The micronisate of tiotropium bromide 1 obtainable by the above method has a characteristic particle size of between 1.0 μm and 3.5 μm, preferably between 1.1 μm and 3.3 μm, most preferably between 1.2 μm and 3.0 μm and Q_{(5.8)} of more than 60%, preferably more than 70%, most preferably more than 80%. The characteristic value Q_{(5.8)} indicates the quantity of particles below 5.8 μm , based on the volume distribution of the particles. The particle sizes were determined within the scope of the present invention by laser diffraction (Fraunhofer diffraction). More detailed information on this subject can be found in the experimental descriptions of the invention.

Also characteristic of the tiotropium micronisate according to the invention which was prepared by the above process are Specific Surface Area values in the range between 2 m²/g and 5 m²/g, more particularly between 2.5 m²/g and 4.5 m²/g and most outstandingly between 3.0 m²/g and 4.0 m²/g.

A particularly preferred aspect of the present invention relates to the inhalable powders according to the invention which are characterised by a content of the tiotropium bromide monohydrate micronisate described hereinbefore as component 1.

For micronising the salmeterol xinafoate 2 which is used according to the invention, the following procedure has proved particularly suitable. The process may be carried out using conventional mills. Preferably, the micronisation is carried out with the exclusion of moisture, more preferably using a corresponding inert gas such as nitrogen, for example. It has proved particularly preferable to use air jet mills in which the material is comminuted by the impact of the particles on one another and on the walls of the grinding container. According to the invention, nitrogen is preferably used as the grinding gas. The material for grinding is conveyed by the grinding gas under specific pressures (grinding pressure). Within the scope of the present invention, the grinding pressure is usually set to a value between about 2 and 12 bar, preferably between about 5 and 10 bar, most preferably between about 5 and 8.5 bar. The material for grinding is fed into the air jet mill by means of the feed gas under specific pressures (feed pressure). Within the scope of the present invention a feed pressure of between about 2 and 12 bar, preferably between about 5.5 and 10.5 bar and most preferably between about 5.5 and 9 bar has proved satisfactory. The feed gas used is also preferably an inert gas, most preferably nitrogen again. The material to be ground (crystalline salmeterol xinafoate) may be fed in at a rate of about 5-100 g/min, preferably at about 10-60 g/min.

A particularly preferred aspect of the present invention relates to the inhalable powders according to the invention which are characterised by a content of micronised salmeterol xinafoate 2 obtained by the micronising process described hereinbefore.

The present invention further relates to the use of the inhalable powders according to the invention for preparing a pharmaceutical composition for the treatment of respiratory diseases, particularly for treating COPD and/or asthma.

The inhalable powders according to the invention may for example be administered using inhalers which meter a single dose from a reservoir by means of a measuring chamber (e.g. according to U.S. Pat. No. 4,570,630A) or by other means (e.g. according to DE 36 25 685 A). Preferably, however, the inhalable powders according to the invention are packed into capsules (to make so-called inhalettes), which are used in inhalers such as those described in WO 94/28958, for example.

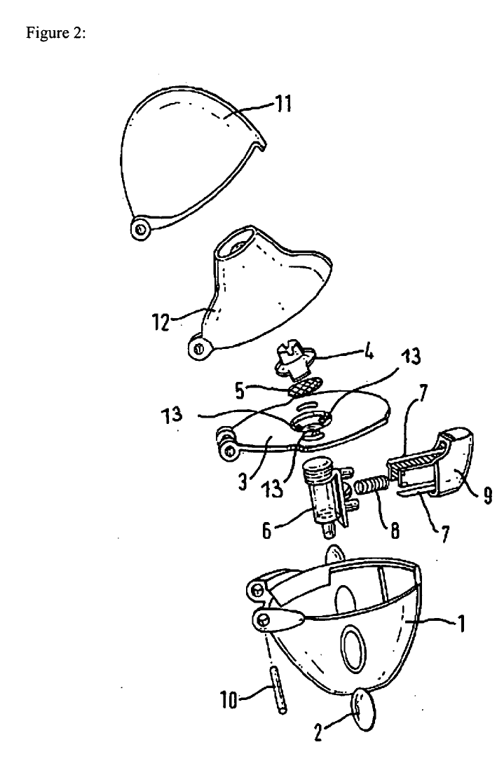

Most preferably, the capsules containing the inhalable powder according to the invention are administered using an inhaler as shown in FIG. 2. This inhaler is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and airholes 13 for adjusting the flow resistance.

The present invention further relates to the use of the inhalable powders according to the invention for preparing a pharmaceutical composition for treating respiratory complaints, particularly for the treatment of COPD and/or asthma, characterised in that the inhaler described above and shown in FIG. 2 is used.

For administering the inhalable powders according to the invention using powder-filled capsules it is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 kg/m³, preferably 940-980 kg/m³, more preferably about 960-970 kg/m³ (high density polyethylene).

The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

In another aspect the present invention relates to the abovementioned capsules which contain the abovementioned inhalable powders according to the invention. These capsules may contain about 1 to 20 mg, preferably about 3 to 15, most preferably about 4 to 12 mg of inhalable powder. Preferred formulations according to the invention contain 4 to 6 mg of inhalable powder. Of equivalent importance according to the invention are capsules for inhalation which contain the formulations according to the invention in an amount of from 8 to 12 mg, more preferably 9 to 11 mg.

The present invention also relates to an inhalation kit consisting of one or more of the above capsules characterised by a content of inhalable powder according to the invention in conjunction with the inhaler according to FIG. 2.

The present invention also relates to the use of the abovementioned capsules characterised by a content of inhalable powder according to the invention, for preparing a pharmaceutical composition for treating respiratory complaints, especially for treating COPD and/or asthma.

Filled capsules which contain the inhalable powders according to the invention are produced by methods known in the art, by filling the empty capsules with the inhalable powders according to the invention.

The following Examples serve to illustrate the present invention in more detail without restricting the scope of the invention to the exemplifying embodiments that follow.

Starting Materials

I) Excipient:

Ia:

In Examples 1 to 24 that follow lactose-monohydrate is used as excipient. It may be obtained for example from Borculo Domo Ingredients, Borculo/NL under the product name Lactochem Extra Fine Powder. The specifications according to the invention for the particle size and specific surface area are met by this grade of lactose. In addition, this lactose has the above-mentioned preferred solution enthalpy values for lactose according to the invention. For example, in the following examples, lactose charges were used which had the following specifications:

a): average particle size: 17.9 μm; 10% fine content: 2.3 μm; specific surface area: 0.61 m²/g; or

b) average particle size: 18.5 μm; 10% fine content: 2.2 μm; specific surface area: 0.83 m²/g;

c) average particle size: 21.6 μm; 10% fine content: 2.5 μm; specific surface area: 0.59 m²/g;

d) average particle size: 16.0 μm; 10% fine content: 2.0 μm; specific surface area: 0.79 m²/g

Ib.:

In Examples 25 to 36 that follow, lactose monohydrate (200 M) is used as the coarser excipient. It may be obtained for example from Messrs DMV International, 5460 VegheU/NL under the product name Pharmatose 200M. This lactose is characterised by an average particle size of about 30 to 35 μm. 200M lactose charges used had an average particle size of 31 μm, for example, with a 10% fine content of 3.2 μm or an average particle size of 34 μm with a 10% fine content of 3.5 μm.

In Examples 25 to 36 that follow, lactose monohydrate with an average particles size of 3-4 μm is used as the finer excipient. It may be obtained by common methods (micronising) from commercially obtainable lactose monohydrate, for example the above-mentioned lactose 200M. Micronised lactose charges used had an average particle size of 3.7 μm, for example, with a 10% fine content of 1.1 μm or an average particle size of 3.2 μm with a 10% fine content of 1.0 μm.

II) Preparation of Salmeterol Xinafoate According to the Invention:

20 g salmeterol base and 9.1 g of 1-hydroxy-2-naphthoic acid are suspended in 260 ml of abs. ethanol and 260 ml tert.butylmethylether. The suspension is heated to 55-56° C. and stirred until a clear solution has formed. The solution is filtered and the filter is rinsed with 30 ml abs. ethanol and 30 ml tert.butylmethylether. The filtrate is cooled to 38° C. and inoculated with a few crystals of salmeterol xinafoate. The solution is stirred for 1 h at 34-37° C., whereupon crystallisation begins. The suspension is cooled to 1-3° C. and stirred for about 30 min at this temperature. The precipitate is removed by suction filtering and washed with 20 ml ethanol and 120 ml tert.butylmethylether. The solid is dried at 45° C. in a nitrogen current. Yield: 26 g (89.5%)

The crystalline salmeterol xinafoate thus obtained has a compacted bulk volume of 0.27 g/cm³.

III) Micronisation of Salmeterol Xinafoate:

The salmeterol xinafoate obtained by the method described above is micronised using an air jet mill of the type MC JETMILL 50 made by Jetpharma; Via Sotto Bisio 42 a/c, 6828-Balerna, Switzerland. Using nitrogen as the grinding gas the following grinding parameters are selected, for example:

grinding pressure 7.5 bar, feed pressure 8.0 bar.

Feed (of crystalline salmeterol xinafoate) or flow rate) 40 g/min.

The micronised salmeterol xinafoate thus obtained has a compacted bulk volume of 0.19 g/cm³.

IV) Micronisation of Crystalline Tiotropium Bromide Monohydrate:

The tiotropium bromide monohydrate obtainable according to WO 02/30928 is micronised with an air jet mill of the 2-inch microniser type with grinding ring, 0.8 mm bore, made by Messrs Sturtevant Inc., 348 Circuit Street, Hanover, Mass. 02239, USA. Using nitrogen as the grinding gas the following grinding parameters are set, for example:

grinding pressure: 5.5 bar; feed pressure: 5.5 bar; supply (of crystalline monohydrate) or flow speed: 19 g/min.

The ground material obtained is then spread out on sheet metal racks in a layer thickness of about 1 cm and subjected to the following climatic conditions for 24-24.5 hours: temperature: 25-30° C.; relative humidity: 70-80%.

Measuring Methods:

I) X-Ray Structural Analysis of Salmeterol Xinafoate:

Measuring Device and Settings:

The X-ray powder diagram was obtained within the scope of the present invention using a BRUKER D8 ADVANCED diffractometer, fitted with a site-sensitive detector (=SSD) and a Cu anode as the X-ray source (CuK_{α}radiation, λ=1.5418 Å, 40 kV, 40 mA).

The X-ray powder diagram obtained for the salmeterol xinafoate according to the invention is shown in FIG. 1. The following Table 1 assembles the data obtained in this spectroscopic analysis:

TABLE 1
Intensities (standardised) of the X-ray reflections

| 2θ [°] | d [Å] | I/Iₒ [%] |
|---|---|---|
| 4.10 | 21.5 | 100 |
| 8.27 | 10.7 | 4 |
| 10.51 | 8.41 | 12 |
| 10.86 | 8.14 | 6 |
| 11.71 | 7.55 | 3 |
| 12.68 | 6.98 | 5 |
| 12.98 | 6.82 | 5 |
| 13.54 | 6.54 | 4 |
| 13.81 | 6.41 | 5 |
| 14.19 | 6.23 | 3 |
| 14.69 | 6.03 | 2 |
| 15.59 | 5.68 | 3 |
| 17.23 | 5.14 | 18 |
| 17.73 | 5.00 | 3 |
| 18.69 | 4.74 | 9 |
| 19.47 | 4.56 | 4 |
| 20.40 | 4.35 | 11 |
| 21.24 | 4.18 | 4 |
| 22.14 | 4.01 | 16 |
| 23.24 | 3.82 | 3 |
| 23.77 | 3.74 | 4 |
| 24.50 | 3.63 | 22 |
| 25.93 | 3.43 | 4 |
| 26.23 | 3.40 | 3 |
| 27.34 | 3.26 | 3 |
| 28.26 | 3.16 | 3 |
| 28.70 | 3.11 | 3 |
| 29.80 | 3.00 | 3 |
| 31.21 | 2.86 | 4 |
| 33.08 | 2.71 | 3 |
| 35.76 | 2.51 | 3 |

In the above Table the value “2 θ[°]” represents the angle of diffraction in degrees and the value “d [Å]” represents the lattice plane spacings determined in Å.

II) Determining the Particle Size of Micronised Tiotropium Monohydrate:

Measuring Equipment and Settings:

The equipment is operated according to the manufacturer's instructions.

| | |
|---|---|
| Measuring equipment: | HELOS Laser-diffraction spectrometer, |
| | (SympaTec) (particle size determined by |
| | Fraunhofer diffraction) |
| Dispersing unit: | RODOS dry disperser with suction funnel, |
| | (SympaTec) |
| Sample quantity: | 200 mg ± 150 mg |
| Product feed: | Vibri Vibrating channel, Messrs. Sympatec |
| Frequency of | rising to 100% |
| vibrating channel: | |
| Duration of sample feed: | 15 to 25 sec. (in the case of 200 mg) |
| Focal length: | 100 mm (measuring range: 0.9-175 μm) |
| Measuring time: | about 15 s (in the case of 200 mg) |
| Cycle time: | 20 ms |
| Start/stop at: | 1% on channel 28 |
| Dispersing gas: | compressed air |
| Pressure: | 3 bar |
| Vacuum: | maximum |
| Evaluation method: | HRLD |

Sample Preparation/Product Feed:

About 200 mg of the test substance are weighed onto a piece of card.

Using another piece of card all the larger lumps are broken up. The powder is then sprinkled finely over the front half of the vibrating channel (starting about 1 cm from the front edge). After the start of the measurement the frequency of the vibrating channel is varied so that the sample is fed in as continuously as possible. However, the quantity of product should not be too great either, so as to ensure adequate dispersal.

III) Determining the Particle Size of Lactose:

Measuring Equipment and Settings:

The equipment is operated according to the manufacturer's instructions.

| | |
|---|---|
| Measuring equipment: | HELOS Laser-diffraction spectrometer, |
| | (SympaTec) (particle size determined by |
| | Fraunhofer diffraction) |
| Dispersing unit: | RODOS dry disperser with suction funnel, |
| | (SympaTec) |
| Sample quantity: | 200 mg ± 100 mg |
| Product feed: | Vibri Vibrating channel, Messrs. Sympatec |
| Frequency of | 100% rising |
| vibrating channel: | |
| Focal length: | 200 mm (measuring range: 1.8-350 μm) |
| Measuring time: | about 10 s (in the case of 200 mg) |
| Cycle time: | 10 ms |
| Start/stop at: | 1% on channel 28 |
| Dispersing gas: | compressed air |
| Pressure: | 3 bar |
| Vacuum: | maximum |
| Evaluation method: | HRLD |

Sample Preparation/Product Feed:

About 200 mg of the test substance are weighed onto a piece of card.

Using another piece of card all the larger lumps are broken up. The powder is transferred into the vibrating channel. A gap of 1.2 to 1.4 mm is set between the vibrating channel and funnel. After the start of the measurement the frequency of the vibrating channel is increased as continuously as possible to 100% towards the end of the measurement.

IV) Determining the Specific Surface Area of Tiotropium Bromide Monohydrate, Micronised (1-Point BET Method):

Method:

The specific surface is determined by exposing the powder sample to a nitrogen/helium atmosphere at different pressures. Cooling the sample causes the nitrogen molecules to be condensed on the surface of the particles. The quantity of condensed nitrogen is determined by means of the change in the thermal heat conductivity of the nitrogen/helium mixture and the surface of the sample is calculated by means of the surface nitrogen requirement. Using this value and the weight of the sample, the specific surface is calculated.

Equipment and Materials:

| | |
|---|---|
| Measuring equipment: | Monosorb, Messrs Quantachrome |
| Heater: | Monotektor, Messrs Quantachrome |
| Measuring and drying gas: | nitrogen (5.0)/helium (4.6) 70/30, Messer |
| | Griesheim |
| Adsorbate: | 30% nitrogen in helium |
| Coolant: | liquid nitrogen |
| Measuring cell: | with capillary tube, Messrs. W. Pabisch |
| | GmbH&Co.KG |
| Calibration peak; | 1000 μl, Messrs. Precision Sampling Corp. |
| Analytical scale: | R 160 P, Messrs. Satorius |

Calculating the Specific Surface:

The measured values are indicated by the equipment in [m²] and are usually converted into [cm²/g] on weighing (dry mass):

$${A}_{spec} = \frac{MW * 10000}{{m}_{tr}}$$

V) Determining the Heat of Solution (Enthalpy of Solution) E_{c}:

The solution enthalpy is determined using a solution calorimeter 2225 Precision Solution Calorimeter made by Messrs. Thermometric.

The heat of solution is calculated by means of the change in temperature occurring (as a result of the dissolving process) and the system-related change in temperature calculated from the base line.

Before and after the ampoule is broken, electrical calibration is carried out with an integrated heating resistor of a precisely known power. A known heat output is delivered to the system over a set period and the jump in temperature is determined.

Measuring Equipment and Settings:

| | |
|---|---|
| Solution calorimeter: | 2225 Precision Solution Calorimeter, |
| | Messrs Thermometric |
| Reaction cell: | 100 ml |
| Thermistor resistance: | 30.0 kΩ (at 25° C.) |
| Speed of stirrer: | 500 U/min |
| Thermostat: | Thermostat of 2277 Thermal Activity Monitor |
| | TAM, Messrs Thermometric |
| Temperature: | 25° C. ± 0.0001° C. (over 24 h) |
| Measuring ampoules: | Crushing ampoules 1 ml, Messrs Thermometric |
| Seal: | Silicon stopper and beeswax, Messrs. |
| | Thermometric |
| Weight: | 40 to 50 mg |
| Solvent: | Chemically pure water |
| Volume of solvent: | 100 ml |
| Bath temperature: | 25° C. |
| Temperature resolution: | High |
| Starting temperature: | −40 mK (±10 mK) temperature-offset |
| Interface: | 2280-002 TAM accessory interface 50 Hz, |
| | Messrs Thermometric |
| Software: | SolCal V 1.1 for WINDOWS |
| Evaluation: | Automatic evaluation with Menu point |
| | CALCULATION/ ANALYSE EXPERIMENT. |
| | (Dynamics of base line; calibration after |
| | breakage of ampoule). |

Electrical Calibration:

The electrical calibration takes place during the measurement, once before and once after the breakage of the ampoule. The calibration after the breakage of the ampoule is used for the evaluation.

| | | |
|---|---|---|
| Amount of heat: | 2.5 | J |
| Heating power: | 500 | mW |
| Heating time: | 10 | s |
| Duration of base lines: | 5 | min (before |
| | | and after heating) |

Preparation of the Powder Formulations According to the Invention:

I) Apparatus

The following machines and equipment, for example, may be used to prepare the inhalable powders:

Mixing container or powder mixer: Turbulamischer 2 L, Type 2C; made by Willy A. Bachofen AG, CH-4500 Basel

Hand-held screen: 0.135 mm mesh size

The empty inhalation capsules may be filled with inhalable powders containing tiotropium by hand or mechanically. The following equipment may be used.

Capsule Filling Machine:

MG2, Type G100, manufacturer: MG2 S.r.l, 1-40065 Pian di Macina di Pianoro (BO), Italy

### EXAMPLE 1

Powder Mixture:

To prepare the powder mixture, 295.43 g of excipient, 0.61 g of micronised tiotropium bromide monohydrate and 3.96 g of micronised salmeterol xinafoate are used. In the resulting 300 g of inhalable powder the content of the active substances are 0.2% of 1′ and 1.32% of 2.

About 40-45 g of excipient are placed in a suitable mixing container through a hand-held screen with a mesh size of 0.315 mm. Then tiotropium bromide monohydrate 1 in batches of about 90-110 mg and excipient in batches of about 40-45 g are screened in in alternate layers. The excipient and active substance 1 are added in 7 and 6 layers, respectively.

Having been screened in, the ingredients are then mixed (mixing speed 900 rpm). The final mixture is passed twice more through a hand-held screen and then mixed again at 900 rpm.

Then using a hand-held screen with a mesh size of 0.315 mm, about 40-45 g of the powder mixture containing the active substance 1 and obtained by the preceding method are placed in a suitable mixing container. Then salmeterol xinafoate 2 in batches of about 650-670 mg and the powder mixture containing the active substance 1 in batches of about 40-45 g are sieved in, in alternate layers. The powder mixture containing the active substance 1 and the active substance 2 are added in 7 and 6 layers, respectively. After being sieved in, the ingredients are mixed (900 rpm). The final mixture is then passed through a hand-held screen twice more and then mixed (900 rpm).

Using the procedure described in Example 1, or analogously thereto, it is possible to obtain inhalable powders which can be packed into suitable plastic capsules to form the following capsules for inhalation, for example:

### EXAMPLE 2

| | |
|---|---|
| tiotropium bromide monohydrate: | 0.0113 mg |
| salmeterol xinafoate | 0.0726 mg |
| lactose monohydrate: | 5.4161 mg |
| polyethylene capsules: | 100.0 mg |
| Total: | 105.5 mg |

### EXAMPLE 3

| | |
|---|---|
| tiotropium bromide monohydrate: | 0.0113 mg |
| salmeterol xinafoate | 0.1450 mg |
| lactose monohydrate: | 5.3437 mg |
| polyethylene capsules: | 100.0 mg |
| Total: | 105.5 mg |

### EXAMPLE 4

| | |
|---|---|
| tiotropium bromide monohydrate: | 0.0225 mg |
| salmeterol xinafoate | 0.1450 mg |
| lactose monohydrate: | 5.3325 mg |
| polyethylene capsules: | 100.0 mg |
| Total: | 105.5 mg |

### EXAMPLE 5

| | |
|---|---|
| tiotropium bromide monohydrate: | 0.0225 mg |
| salmeterol xinafoate | 0.2180 mg |
| lactose monohydrate: | 10.7595 mg |
| polyethylene capsules: | 100.0 mg |
| Total: | 111.0 mg |

### EXAMPLE 6

| | |
|---|---|
| tiotropium bromide monohydrate: | 0.0056 mg |
| salmeterol xinafoate | 0.0726 mg |
| lactose monohydrate: | 5.4218 mg |
| polyethylene capsules: | 100.0 mg |
| Total: | 105.5 mg |

### EXAMPLE 7

| | |
|---|---|
| tiotropium bromide monohydrate: | 0.0056 mg |
| salmeterol xinafoate | 0.1090 mg |
| lactose monohydrate: | 5.3854 mg |
| polyethylene capsules: | 100.0 mg |
| Total: | 105.5 mg |

### EXAMPLE 8

| | |
|---|---|
| tiotropium bromide monohydrate: | 0.0125 mg |
| salmeterol xinafoate | 0.0363 mg |
| lactose monohydrate: | 9.9512 mg |
| polyethylene capsules: | 100.0 mg |
| Total: | 110.0 mg |

### EXAMPLE 9

| | |
|---|---|
| tiotropium bromide monohydrate: | 0.0125 mg |
| salmeterol xinafoate | 0.0435 mg |
| lactose monohydrate: | 9.9440 mg |
| polyethylene capsules: | 100.0 mg |
| Total: | 110.0 mg |

### EXAMPLE 10

| | |
|---|---|
| tiotropium bromide monohydrate: | 0.0125 mg |
| salmeterol xinafoate | 0.0508 mg |
| lactose monohydrate: | 9.9367 mg |
| polyethylene capsules: | 100.0 mg |
| Total: | 110.0 mg |

### EXAMPLE 11

| | |
|---|---|
| tiotropium bromide monohydrate: | 0.0225 mg |
| salmeterol xinafoate | 0.0435 mg |
| lactose monohydrate: | 9.9340 mg |
| polyethylene capsules: | 100.0 mg |
| Total: | 110.0 mg |

### EXAMPLE 12

| | |
|---|---|
| tiotropium bromide monohydrate: | 0.0063 mg |
| salmeterol xinafoate | 0.0435 mg |
| lactose monohydrate: | 9.9502 mg |
| polyethylene capsules: | 100.0 mg |
| Total: | 110.0 mg |

### EXAMPLE 13

Powder Mixture:

To prepare the powder mixture, 295.43 g of excipient, 0.61 g of micronised tiotropium bromide monohydrate and 3.96 g of micronised salmeterol xinafoate are used. In the resulting 300 g of inhalable powder the content of the active substances are 0.2% of 1′ and 1.32% of 2.

About 20-23 g of excipient are placed in a suitable mixing container through a hand-held screen with a mesh size of 0.315 mm. Then tiotropium bromide monohydrate 1 in batches of about 90-110 mg and excipient in batches of about 40-45 g and salmeterol xinafoate in batches of about 650-670 mg are screened in in alternate layers. This procedure is repeated 6 times. Finally, a last batch of excipient of about 20-23 g is added.

Having been screened in, the ingredients (6 layers each of 1 and 2 and 13 layers of excipient) are then mixed (mixing speed 900 rpm). The final mixture is passed twice more through a hand-held screen and then mixed again at 900 rpm.

Using the procedure described in Example 13, or analogously thereto, it is possible to obtain inhalable powders which can be packed into suitable plastic capsules to form the following capsules for inhalation, for example:

### EXAMPLE 14

| | |
|---|---|
| tiotropium bromide monohydrate: | 0.0113 mg |
| salmeterol xinafoate | 0.0726 mg |
| lactose monohydrate: | 5.4161 mg |
| polyethylene capsules: | 100.0 mg |
| Total: | 105.5 mg |

### EXAMPLE 15

| | |
|---|---|
| tiotropium bromide monohydrate: | 0.0113 mg |
| salmeterol xinafoate | 0.1450 mg |
| lactose monohydrate: | 5.3437 mg |
| polyethylene capsules: | 100.0 mg |
| Total: | 105.5 mg |

### EXAMPLE 16

| | |
|---|---|
| tiotropium bromide monohydrate: | 0.0225 mg |
| salmeterol xinafoate | 0.1450 mg |
| lactose monohydrate: | 5.3325 mg |
| polyethylene capsules: | 100.0 mg |
| Total: | 105.5 mg |

### EXAMPLE 17

| | |
|---|---|
| tiotropium bromide monohydrate: | 0.0225 mg |
| salmeterol xinafoate | 0.2180 mg |
| lactose monohydrate: | 10.7595 mg |
| polyethylene capsules: | 100.0 mg |
| Total: | 111.0 mg |

### EXAMPLE 18

| | |
|---|---|
| tiotropium bromide monohydrate: | 0.0056 mg |
| salmeterol xinafoate | 0.0726 mg |
| lactose monohydrate: | 5.4218 mg |
| polyethylene capsules: | 100.0 mg |
| Total: | 105.5 mg |

### EXAMPLE 19

| | |
|---|---|
| tiotropium bromide monohydrate: | 0.0056 mg |
| salmeterol xinafoate | 0.1090 mg |
| lactose monohydrate: | 5.3854 mg |
| polyethylene capsules: | 100.0 mg |
| Total: | 105.5 mg |

### EXAMPLE 20

| | |
|---|---|
| tiotropium bromide monohydrate: | 0.0125 mg |
| salmeterol xinafoate | 0.0363 mg |
| lactose monohydrate: | 9.9512 mg |
| polyethylene capsules: | 100.0 mg |
| Total: | 110.0 mg |

### EXAMPLE 21

| | |
|---|---|
| tiotropium bromide monohydrate: | 0.0125 mg |
| salmeterol xinafoate | 0.0435 mg |
| lactose monohydrate: | 9.9440 mg |
| polyethylene capsules: | 100.0 mg |
| Total: | 110.0 mg |

### EXAMPLE 22

| | |
|---|---|
| tiotropium bromide monohydrate: | 0.0125 mg |
| salmeterol xinafoate | 0.0508 mg |
| lactose monohydrate: | 9.9367 mg |
| polyethylene capsules: | 100.0 mg |
| Total: | 110.0 mg |

### EXAMPLE 23

| | |
|---|---|
| tiotropium bromide monohydrate: | 0.0225 mg |
| salmeterol xinafoate | 0.0435 mg |
| lactose monohydrate: | 9.9340 mg |
| polyethylene capsules: | 100.0 mg |
| Total: | 110.0 mg |

### EXAMPLE 24

| | |
|---|---|
| tiotropium bromide monohydrate: | 0.0063 mg |
| salmeterol xinafoate | 0.0435 mg |
| lactose monohydrate: | 9.9502 mg |
| polyethylene capsules: | 100.0 mg |
| Total: | 110.0 mg |

### EXAMPLE 25

Powder Mixture:

To prepare the powder mixture, 295.43 g of excipient, 0.61 g of micronised tiotropium bromide monohydrate and 3.96 g of micronised salmeterol xinafoate are used. In the resulting 300 g of inhalable powder the content of the active substances are 0.2% of 1′ and 1.32% of 2.

The excipient used is a mixture of 280.43 g of the lactose monohydrate mentioned under point Ib with 15 g of the micronised lactose monohydrate mentioned under point Ib with an average particle size of about 3-4 μm. In the resulting pharmaceutical formulation the proportion of excipient fraction with the smaller average particle size is 5%.

About 29-33 g of coarser excipient are placed in a suitable mixing container through a hand-held screen with a mesh size of 0.315 mm. Then about 1.5-2 g of finer excipient are screened in in layers. This procedure is repeated 8 times. Finally, a last batch of coarser excipient of about 29-33 g is added.

Having been screened in, the ingredients (9 layers of excipient with a coarser average particle size and 8 layers of micronised excipient) are mixed together (mixing speed 900 rpm).

The excipient mixture thus obtained is then subjected to the procedure according to Example 13 in order to prepare the final mixture. The ingredients sieved in (6 layers each of 1 and 2 and 13 layers of excipient mixture) are then mixed together (mixing speed 900 rpm). The final mixture is passed through a hand-held screen twice more and the mixed (mixing speed 900 rpm).

Using the procedure described in Example 25, or analogously thereto, it is possible to obtain inhalable powders which can be packed into suitable plastic capsules to form the following capsules for inhalation, for example. In the Examples that follow, the term lactose monohydrate (3-4 μm) denotes micronised lactose and the term lactose monohydrate denotes coarser lactose:

### EXAMPLE 26

| | |
|---|---|
| tiotropium bromide monohydrate: | 0.0113 mg |
| salmeterol xinafoate | 0.0726 mg |
| lactose monohydrate (3-4 μm): | 0.2750 mg |
| lactose monohydrate: | 5.1411 mg |
| polyethylene capsules: | 100.0 mg |
| Total: | 105.5 mg |

### EXAMPLE 27

| | |
|---|---|
| tiotropium bromide monohydrate: | 0.0113 mg |
| salmeterol xinafoate | 0.1450 mg |
| lactose monohydrate (3-4 μm): | 0.2750 mg |
| lactose monohydrate: | 5.0687 mg |
| polyethylene capsules: | 100.0 mg |
| Total: | 105.5 mg |

### EXAMPLE 28

| | |
|---|---|
| tiotropium bromide monohydrate: | 0.0225 mg |
| salmeterol xinafoate | 0.1450 mg |
| lactose monohydrate (3-4 μm): | 0.2750 mg |
| lactose monohydrate: | 5.0575 mg |
| polyethylene capsules: | 100.0 mg |
| Total: | 105.5 mg |

### EXAMPLE 29

| | |
|---|---|
| tiotropium bromide monohydrate: | 0.0225 mg |
| salmeterol xinafoate | 0.2180 mg |
| lactose monohydrate (3-4 μm): | 0.5500 mg |
| lactose monohydrate: | 10.2095 mg |
| polyethylene capsules: | 100.0 mg |
| Total: | 111.0 mg |

### EXAMPLE 30

| | |
|---|---|
| tiotropium bromide monohydrate: | 0.0056 mg |
| salmeterol xinafoate | 0.0726 mg |
| lactose monohydrate (3-4 μm): | 0.2750 mg |
| lactose monohydrate: | 5.1468 mg |
| polyethylene capsules: | 100.0 mg |
| Total: | 105.5 mg |

### EXAMPLE 31

| | |
|---|---|
| tiotropium bromide monohydrate: | 0.0056 mg |
| salmeterol xinafoate | 0.1090 mg |
| lactose monohydrate (3-4 μm): | 0.2750 mg |
| lactose monohydrate: | 5.1104 mg |
| polyethylene capsules: | 100.0 mg |
| Total: | 105.5 mg |

### EXAMPLE 32

| | |
|---|---|
| tiotropium bromide monohydrate: | 0.0125 mg |
| salmeterol xinafoate | 0.0363 mg |
| lactose monohydrate (3-4 μm): | 0.5000 mg |
| lactose monohydrate: | 9.4512 mg |
| polyethylene capsules: | 100.0 mg |
| Total: | 110.0 mg |

### EXAMPLE 33

| | |
|---|---|
| tiotropium bromide monohydrate: | 0.0125 mg |
| salmeterol xinafoate | 0.0435 mg |
| lactose monohydrate (3-4 μm): | 0.5000 mg |
| lactose monohydrate: | 9.4440 mg |
| polyethylene capsules: | 100.0 mg |
| Total: | 110.0 mg |

### EXAMPLE 34

| | |
|---|---|
| tiotropium bromide monohydrate: | 0.0125 mg |
| salmeterol xinafoate | 0.0508 mg |
| lactose monohydrate (3-4 μm): | 0.5000 mg |
| lactose monohydrate: | 9.4367 mg |
| polyethylene capsules: | 100.0 mg |
| Total: | 110.0 mg |

### EXAMPLE 35

| | |
|---|---|
| tiotropium bromide monohydrate: | 0.0225 mg |
| salmeterol xinafoate | 0.0435 mg |
| lactose monohydrate (3-4 μm): | 0.5000 mg |
| lactose monohydrate: | 9.4340 mg |
| polyethylene capsules: | 100.0 mg |
| Total: | 110.0 mg |

### EXAMPLE 36

| | |
|---|---|
| tiotropium bromide monohydrate: | 0.0063 mg |
| salmeterol xinafoate | 0.0435 mg |
| lactose monohydrate (3-4 μm): | 0.5000 mg |
| lactose monohydrate: | 9.4502 mg |
| polyethylene capsules: | 100.0 mg |
| Total: | 110.0 mg |

1) a composition comprising tiotropium, or a pharmaceutically acceptable salt thereof, salmeterol xinafoate, and a physiologically acceptable excipient:

2) The composition according to claim 1 comprising tiotropium in combination with a counter-ion selected from among the chloride, bromide, iodide, methanesulphonate or para-toluenesulphonate thereof.

3) The composition according to claim 1 wherein the salmeterol xinafoate comprises the characteristic values d=21.5 Å; 8.41 Å; 5.14 Å; 4.35 Å; 4.01 Å and 3.63 Å in an X-ray powder diagram.

4) The composition according to claim 1 wherein the salmeterol xinafoate has a compacted bulk volume of ≧0.134 g/cm³.

5) The composition according to claim 1 comprising salmeterol xinafoate in an amount of 0.002 to 15%.

6) The composition according to claim 1 comprising tiotropium in an amount of 0.001 to 5%.

7) The composition according to claim 1 comprising tiotropium and salmeterol xinafoate in a combined amount of from 5 to 5000 μg.

8) The composition according to claim 1 comprising a physiologically acceptable excipient selected from the group consisting of monosaccharides, disaccharides, oligo- and polysaccharides, polyalcohols and salts.

9) The composition according to claim 8 wherein the physiologically acceptable excipient is selected from among glucose, arabinose, lactose, saccharose, maltose and trehalose, optionally in the form of the hydrates thereof.

10) The composition according to claim 1 wherein the salmeterol xinafoate has a melting point of about 124° C.

11) A capsule comprising the composition according to claim 1.

12) The capsule according to claim 11 comprising 1 to 20 mg of the composition.

13) The capsule according to claim 12 comprising 4 to 6 mg of the composition.

14) The capsule according to claim 12 comprising 8 to 12 mg of the composition.

15) An inhalation kit comprising the capsule according to claim 11 and an inhaler.

16) The inhalation kit according to claim 15 wherein the inhaler comprises a housing comprising two windows, a deck in which there are air inlet ports and which is provided with a screen secured via a screen housing, an inhalation chamber connected to the deck on which there is a push button provided with two sharpened pins and movable counter to a spring, and a mouthpiece which is connected to the housing, the deck and a cover via a spindle to enable it to open or shut, and airholes for adjusting the flow resistance.

17) The composition according to claim 1 in the form of an inhalable powder.

18) The composition according to claim 2 in the form of an inhalable powder.

19) The composition according to claim 5 in the form of an inhalable powder.

20) The composition according to claim 6 in the form of an inhalable powder.
